(19) United States
(12) Patent Application Publication
(10) Pub. No.: US 2022/0291236 A1
(43) Pub. Date: Sep. 15, 2022

(54) DETECTION OF DYSTROGLYCAN

(71) Applicant: ML Bio Solutions Inc., Charlotte, NC (US)

(72) Inventor: Hector M. RODRIGUEZ, San Bruno, CA (US)

(21) Appl. No.: 17/679,841

(22) Filed: Feb. 24, 2022

Related U.S. Application Data
(60) Provisional application No. 63/154,435, filed on Feb. 26, 2021.

(51) Int. Cl. G01N 33/68; C07K 16/18

(52) U.S. Cl. CPC G01N 33/6893; C07K 16/18; G01N 2333/47; G01N 2333/78

(57) ABSTRACT

Provided are methods of determining an amount of alpha-dystroglycan (αDG) in a sample, determining an amount of the glycosylated form of αDG in the sample, and determining a ratio of the amount of the glycosylated form of αDG to the amount of αDG in the sample.

### RELATED APPLICATIONS

This application claims priority to, and the benefit of, U.S. Provisional Application No. 63/154,435, filed Feb. 26, 2021. The contents of the aforementioned patent application are incorporated herein by reference in its entirety.

### BACKGROUND

In healthy muscle cells, the sugar chain on the protein of alpha-dystroglycan (αDG) contains tandem structures of ribitol-phosphate, a pentose alcohol that was previously unknown in humans. The genes fukutin (FKTN), fukutin-related protein (FKRP), and isoprenoid synthase domain-containing protein (ISPD) encode essential enzymes for the synthesis of this structure. ISPD metabolically converts ribitol-5-phosphate into CDP-ribitol, a substrate for fukutin and FKRP. Subsequently, fukutin transfers a first ribitol-phosphate onto sugar chains of αDG followed by FKRP that transfers a subsequent ribitol-phosphate. Abnormal glycosylation of αDG is associated with a range of neurological and physical impairments. A variety of dystroglycanopathies including Walker-Warburg Syndrome, Muscle Eye Brain Disease, Fukuyama Congenital Muscular Dystrophy (CMD), and Limb Girdle Muscular Dystrophies have been identified and are generally characterized by different genetic mutations. Limb-Girdle Muscular Dystrophy (LGMD2i), also known as LGMD R9, is a dystroglycanopathy caused by partial loss of function mutations in the FKRP gene.

Heng et al. discloses methods of detecting αDG in human urine using a sandwich ELISA technique (Heng et al., J. Biomolec Screen, 4(21); 2015). However, simultaneous detection of αDG and glycosylated αDG is needed for rapid and consistent quantification of the extent of αDG glycosylation to keep pace with the dynamic nature of this biochemical process.

There remains an unmet need for methods of simultaneous detection of αDG and glycosylated αDG for dystroglycanopathy patient biopsies.

### SUMMARY

The present disclosure provides multiplexed analytical methods for simultaneous detection of alpha-dystroglycan (αDG) and glycosylated αDG in a sample. These methods can be used to determine a ratio of glycosylated αDG to total αDG utilizing, e.g., signals obtained via imaging of a membrane. Such methods provide a mechanism for assessing the glycosylation of αDG in cells and tissue samples from subjects (e.g., patients) with dystroglycanopathies (e.g., Walker-Warburg Syndrome, Muscle Eye Brain Disease, Fukuyama Congenital Muscular Dystrophy (CMD), and Limb Girdle Muscular Dystrophies) to, for example, assess longitudinal disease progression. Due to the ratio-metric nature of these methods, the methods can also be used to evaluate the impact of therapeutics that seek to restore the glycosylation of αDG and may assist in guiding dose selection.

Accordingly, in an aspect, the present disclosure relates to a method of determining an amount of alpha-dystroglycan (αDG) in a sample, determining an amount of the glycosylated form of αDG in the sample, and determining a ratio of the amount of the glycosylated form of αDG to the amount of αDG in the sample.

In some embodiments, determining an amount of alpha-dystroglycan (αDG) in a sample and determining an amount of the glycosylated form of αDG in the sample are performed simultaneously.

In some embodiments, determining an amount of alpha-dystroglycan (αDG) in a sample and determining an amount of the glycosylated form of αDG in the sample comprise performing a Western Blotting analytical method (e.g., as described herein). In some embodiments, a Western Blotting analytical method comprises performing gel electrophoresis.

In some embodiments, determining an amount of alpha-dystroglycan (αDG) in a sample and determining an amount of the glycosylated form of αDG in the sample comprises contacting the sample with one or more antibodies.

In some embodiments, the one or more antibodies comprise one or more monoclonal antibodies. In some embodiments, the one or more antibodies comprise one or more polyclonal antibodies.

In some embodiments, an antibody of the one or more antibodies is used to determine an amount of αDG and/or an amount of the glycosylated form of αDG having a molecular weight of between about 50 kiloDaltons (kDa) and about 260 kDa.

In some embodiments, an antibody of the one or more antibodies is used to determine an amount of αDG and/or an amount of the glycosylated form of αDG having a molecular weight of between about 125 kDa and about 260 kDa.

In some embodiments, determining an amount of alpha-dystroglycan (αDG) in a sample comprises contacting the sample with an anti-αDG antibody and determining an amount of the glycosylated form of αDG in the sample comprises contacting the sample with a matriglycan-specific αDG antibody. In some embodiments, the anti-αDG antibody is AF6868 alpha-dystroglycan antibody. In some embodiments, the matriglycan-specific αDG antibody is IIH6C4 alpha-dystroglycan antibody.

In some embodiments, the sample is a biopsy sample. In some embodiments, the sample is derived from muscle tissue. In some embodiments, the sample is derived from a subject. In some embodiments, the subject is a human subject.

In some embodiments, the subject has been diagnosed with a dystroglycanopathy. In some embodiments, the subject has been diagnosed with Walker-Warburg Syndrome, Muscle Eye Brain Disease, Fukuyama Congenital Muscular Dystrophy (CMD), or a Limb Girdle Muscular Dystrophy. In some embodiments, the subject has been diagnosed with limb girdle muscular dystrophy type 2i (LGMD2i). In some embodiments, the subject is suspected to have a dystroglycanopathy. In some embodiments, the subject is suspected to have limb girdle muscular dystrophy type 2i (LGMD2i).

In some embodiments, the method further comprises, based at least in part on determining a ratio of the amount of the glycosylated form of αDG to the amount of αDG in the sample, determining that the subject has a dystroglycanopathy (e.g., Walker-Warburg Syndrome, Muscle Eye Brain Disease, Fukuyama Congenital Muscular Dystrophy (CMD), or a Limb Girdle Muscular Dystrophy).

In some embodiments, the subject is determined to have limb girdle muscular dystrophy type 2i (LGMD2i). In some embodiments, the subject is suspected to have a dystroglycanopathy. In some embodiments, the subject is suspected to have LGMD2i.

In some embodiments, the method further comprises, based at least in part on determining a ratio of the amount of the glycosylated form of αDG to the amount of αDG in the sample, providing a recommendation to administer a therapeutic agent to the subject.

In some embodiments, the therapeutic agent is ribitol or a form thereof.

In another aspect, the present disclosure relates to a method of determining the amount of core alpha-dystroglycan (αDG) protein in a sample, wherein the core αDG protein is specifically recognized by an anti-αDG antibody, determining the amount of an additional αDG population in the sample, wherein the additional αDG population is specifically recognized by a matriglycan-specific αDG antibody, and determining a ratio between the amount of the core αDG protein and the amount of the additional αDG population. In some embodiments, the additional αDG population comprises a glycosylated form of αDG.

In some embodiments, determining the amount of core alpha-dystroglycan (αDG) protein in a sample, wherein the core αDG protein is specifically recognized by an anti-αDG antibody and determining the amount of an additional αDG population in the sample, wherein the additional αDG population is specifically recognized by a matriglycan-specific αDG antibody are performed simultaneously.

In some embodiments, determining the amount of core alpha-dystroglycan (αDG) protein in a sample, wherein the core αDG protein is specifically recognized by an anti-αDG antibody and determining the amount of an additional αDG population in the sample, wherein the additional αDG population is specifically recognized by a matriglycan-specific αDG antibody comprise performing a Western Blotting analytical method (e.g., as described herein). In some embodiments, a Western Blotting analytical method comprises performing gel electrophoresis.

In some embodiments, the anti-αDG antibody and/or the matriglycan-specific αDG antibody is a polyclonal antibody.

In some embodiments, the anti-αDG antibody and/or the matriglycan-specific αDG antibody is a monoclonal antibody.

In some embodiments, determining the amount of core alpha-dystroglycan (αDG) protein in a sample comprises contacting the sample with the anti-αDG antibody and determining the amount of an additional αDG population in the sample comprises contacting the sample with the matriglycan-specific αDG antibody.

In some embodiments, the anti-αDG antibody is used to determine an amount of αDG having a molecular weight of between about 50 kDa and about 260 kDa.

In some embodiments, the matriglycan-specific αDG antibody is used to determine an amount of the glycosylated form of αDG having a molecular weight of greater than 50 KDa.

In some embodiments, the sample for implementing a method of determining the amount of core alpha-dystroglycan and a glycosylated form of αDG is a biopsy sample. In some embodiments, the sample is derived from muscle tissue. In some embodiments, the sample is derived from a subject. In some embodiments, the subject is a human subject.

In some embodiments, the subject has been diagnosed with a dystroglycanopathy. In some embodiments, the subject has been diagnosed with Walker-Warburg Syndrome, Muscle Eye Brain Disease, Fukuyama Congenital Muscular Dystrophy (CMD), or a Limb Girdle Muscular Dystrophy. In some embodiments, the subject has been diagnosed with limb girdle muscular dystrophy type 2i (LGMD2i).

In some embodiments, the method further comprises, based at least in part on determining a ratio of the amount of the glycosylated form of αDG to the amount of αDG in the sample, determining that the subject has a dystroglycanopathy. In some embodiments, the dystroglycanopathy is Walker-Warburg Syndrome, Muscle Eye Brain Disease, Fukuyama Congenital Muscular Dystrophy (CMD), or a Limb Girdle Muscular Dystrophy.

In some embodiments, the subject is determined to have limb girdle muscular dystrophy type 2i (LGMD2i). In some embodiments, the subject is suspected to have a dystroglycanopathy. In some embodiments, the subject is suspected to have LGMD2i.

In some embodiments, the method further comprises, based at least in part on determining a ratio between the amount of the core αDG protein and the amount of the additional αDG population, determining that the subject has a dystroglycanopathy. In some embodiments, the dystroglycanopathy is Walker-Warburg Syndrome, Muscle Eye Brain Disease, Fukuyama Congenital Muscular Dystrophy (CMD), or a Limb Girdle Muscular Dystrophy. In some embodiments, the dystroglycanopathy is LGMD2i.

In some embodiments, the method further comprises, based at least in part on determining a ratio between the amount of the core αDG protein and the amount of the additional αDG population, providing a recommendation to administer a therapeutic agent to the subject. In some embodiments, the therapeutic agent is ribitol or a form thereof.

In one aspect, the present disclosure relates to a method of providing a first sample from a subject having a first ratio of an amount of a glycosylated form of alpha-dystroglycan (αDG) to an amount of αDG in the first sample, providing a second sample from the subject having a second ratio of an amount of a glycosylated form of αDG to an amount of αDG in the second sample, and determining a difference between the first ratio and the second ratio.

In some embodiments, the first sample was collected from the subject at a first timepoint and the second sample was collected from the subject at a second timepoint, which second timepoint is later than the first timepoint.

In some embodiments, the first sample was collected from the subject prior to the subject undergoing a treatment regimen for a dystroglycanopathy. In some embodiments, the dystroglycanopathy is Walker-Warburg Syndrome, Muscle Eye Brain Disease, Fukuyama Congenital Muscular Dystrophy (CMD), or a Limb Girdle Muscular Dystrophy. In some embodiments, the dystroglycanopathy is limb girdle muscular dystrophy type 2i (LGMD2i). In some embodiments, the treatment regimen comprises administration of ribitol or a form thereof.

In some embodiments, the second sample was collected from the subject while the subject is undergoing a treatment regimen for a dystroglycanopathy. In some embodiments, the dystroglycanopathy is Walker-Warburg Syndrome, Muscle Eye Brain Disease, Fukuyama Congenital Muscular Dystrophy (CMD), or a Limb Girdle Muscular Dystrophy. In some embodiments, the dystroglycanopathy is limb girdle muscular dystrophy type 2i (LGMD2i). In some embodiments, the treatment regimen comprises administration of ribitol or a form thereof.

In some embodiments, the second sample was collected from the subject after the subject has undergone a treatment regimen for a dystroglycanopathy. In some embodiments, the dystroglycanopathy is Walker-Warburg Syndrome, Muscle Eye Brain Disease, Fukuyama Congenital Muscular Dystrophy (CMD), or a Limb Girdle Muscular Dystrophy. In some embodiments, the dystroglycanopathy is limb girdle muscular dystrophy type 2i (LGMD2i). In some embodiments, the treatment regimen comprises administration of ribitol or a form thereof.

In some embodiments, the second timepoint is at least 1 week after the first timepoint. In some embodiments, the second timepoint is at least 1 month after the first timepoint. In some embodiments, the second timepoint is at least 3 months after the first timepoint. In some embodiments, the second timepoint is at least 6 months after the first timepoint. In some embodiments, the second timepoint is at least 1 year after the first timepoint.

In some embodiments, the methods of the present disclosure comprise collecting the first sample and/or the second sample from the subject.

In some embodiments, the methods of the present disclosure comprise determining a relative amount of alpha-dystroglycan (αDG) in the first and/or second sample, determining a relative amount of the glycosylated form of αDG in the first and/or second sample, and determining the first and/or second ratio of the relative amount of the glycosylated form of αDG to the relative amount of αDG in the first and/or second sample.

In some embodiments, determining a relative amount of alpha-dystroglycan (αDG) in the first and/or second sample and determining a relative amount of the glycosylated form of αDG in the first and/or second sample comprises performing a Western Blotting analytical method (e.g., as described herein). In some embodiments, a Western Blotting analytical method comprises performing gel electrophoresis.

In some embodiments, determining a relative amount of alpha-dystroglycan (αDG) in the first and/or second sample and determining a relative amount of the glycosylated form of αDG in the first and/or second sample comprises contacting the first and/or second sample with one or more antibodies.

In some embodiments, the one or more antibodies comprises one or more monoclonal antibodies. In some embodiments, the one or more antibodies comprise one or more polyclonal antibodies.

In some embodiments, the one or more antibodies used to determine an amount of αDG and/or an amount of the glycosylated form of αDG has a molecular weight of between about 50 kDa and about 260 kDa.

In some embodiments, the one or more antibodies used to determine an amount of αDG and/or an amount of the glycosylated form of αDG has a molecular weight of between about 125 kDa and about 260 kDa.

In some embodiments, the method comprises determining a relative amount of alpha-dystroglycan (αDG) in the first and/or second sample comprises contacting the first and/or second sample with an anti-αDG antibody and determining a relative amount of the glycosylated form of αDG in the first and/or second sample comprises contacting the first and/or second sample with a matriglycan-specific αDG antibody.

In some embodiments, the anti-αDG antibody is AF6868 alpha-dystroglycan antibody.

In some embodiments, the matriglycan-specific αDG antibody is IIH6C4 alpha-dystroglycan antibody.

In some embodiments, the method further comprises, based at least in part on determining the first and/or second ratio of the relative amount of the glycosylated form of αDG to the relative amount of αDG in the first and/or second sample, providing a recommendation to administer a therapeutic agent to the subject. In some embodiments, the therapeutic agent is ribitol or a form thereof.

In some embodiments, the method further comprises administering a therapeutic agent to the subject. In some embodiments, the therapeutic agent is ribitol or a form thereof.

In some embodiments, the method further comprises, based at least in part on determining the first and/or second ratio of the relative amount of the glycosylated form of αDG to the relative amount of αDG in the first and/or second sample, providing a recommendation to change a dosing regimen of a therapeutic agent for the subject. In some embodiments, the therapeutic agent is ribitol or a form thereof.

In some embodiments, the recommendation comprises a recommendation to increase dosing of the therapeutic agent. In some embodiments, the recommendation comprises a recommendation to decrease dosing of the therapeutic agent. In some embodiments, the therapeutic agent is ribitol or a form thereof.

In some embodiments, the first sample and the second sample are of a same type. In some embodiments, the first sample and the second sample are biopsy samples. In some embodiments, the first sample and the second sample are derived from muscle tissue.

### BRIEF DESCRIPTION OF FIGURES

FIG. 1 is a diagram of the model for ribitol-induced functional glycosylation of αDG in FKRP mutant cells. ?=a mechanism is not fully understood; “*”=the first ribitol-5-phospate on the Core M3 of αDG is transferred by fukutin using CDP-ribitol as the donor substrate; CTP=Cytidine Triphosphate; D-Glucuronic acid (GlcA); Xylitol (Xyl); N-Acetyl-D-galactosamine (GalNAc); N-Acetyl-D-glucosamine (GlcNAc); D-Mannose (Man) (Source: Cataldi et al. 2018)

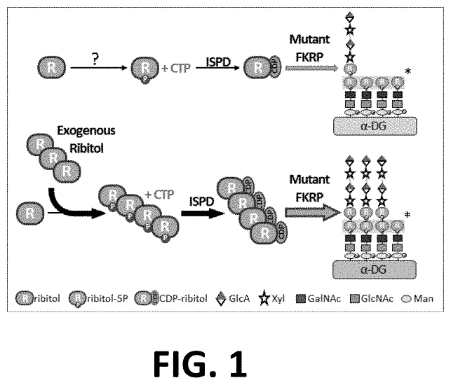

FIG. 2A is a graph depicting the linearity of each detection channel as a function of lysate concentration for serially diluted gastrocnemius tissue lysate. The solid line represents the linear regression.

FIG. 2B is an image of a Western Blot probed for total α-dystroglycan (800 nanometers (nm)—green) and glycosylated α-dystroglycan (700 nm—red) by multiplexed LI-COR and displays the merge of both detection channels (yellow) with regions of interest for αDG (˜50-260 kDa) and a putative N-terminal fragment (˜27 kDa) indicated with yellow boxes. Red * denotes a sample that appeared to be predominantly adipose rather than muscle during tissue processing.

FIG. 2C is a graph depicting the ratio of glycosylated αDG to total αDG protein determined using intensities from the detection channels shown in FIG. 2B and plotted for each muscle type. The plot shows the mean and the standard deviation of the ratios.

FIG. 3A is an image of a Western Blot probed for total α-dystroglycan (800 nm—green) and glycosylated α-dystroglycan (700 nm—red) by multiplexed LI-COR and displays the merge of both detection channels (yellow) in healthy and mutant (P488L and L276I) mouse heart and gastrocnemius tissues.

FIG. 3B is an image of a Western Blot probed for total α-dystroglycan (800 nm—green) and glycosylated α-dystroglycan (700 nm—red) by multiplexed LI-COR and displays the merge of both detection channels (yellow) in healthy and mutant (P488L and L276I) mouse diaphragm and tibialis anterior (TA) tissues.

FIG. 3C is a graph depicting the ratio of total αDG to total protein determined using intensities from the detection channels shown in FIGS. 3A and 3B and plotted for each muscle type. All mutants were considered as a single dataset for the statistical analysis. The plot shows the mean and the standard deviation of the ratios. Statistical significance was determined by t-test: *: p=0.05-0.01; ***: p=0.001-0.0001; ****: p<0.0001

FIG. 3D is a graph depicting the ratio of glycosylated αDG to total αDG determined using intensities from the detection channels shown in FIGS. 3A and 3B and plotted for each muscle type. All mutants were considered as a single dataset for the statistical analysis. The plot shows the mean and the standard deviation of the ratios. Statistical significance was determined by t-test: *: p=0.05-0.01; ***: p=0.001-0.0001; ****: p<0.0001

FIG. 4 is a graph depicting the ratio of glycosylated αDG to total αDG assessed in gastrocnemius muscle tissue from a healthy human donor (Healthy Human) and a wild type mouse (WT-Mouse), TA muscle tissue from a L276I FKRP mutant mouse model (L276I Mouse) and a P448L mutant mouse model (P448L Mouse), and three LGMD2I patients containing a L276I FRKP mutation (two homozygous: [P1 L276I and P3 L276I] and one compound heterozygous: [P2 L276I]).

FIG. 5 is a graph depicting the ratio of glycosylated αDG to total αDG assessed in human LGMD2I patients with mutation L276I. The points on the plot represent the average of triplicate analysis for each patient. Shown are the mean and standard deviation for the healthy controls, L276I FKRP homozygous and compound heterozygous patients. The asterisks denote statistical significance. (HOM p=0.0125, HET p=0.0049 relative to control)

FIG. 6A is an image of a Western Blot probed for total α-dystroglycan (800 nm—green) and glycosylated α-dystroglycan (700 nm—red) by multiplexed LI-COR and displays the merge of both detection channels (yellow) with regions of interest for αDG (˜50-250 kDa) indicated with yellow boxes. The samples included healthy heart muscle tissue, healthy gastrocnemius muscle tissue, and P448L mutant FKRP heart and gastrocnemius tissues that were untreated or treated with 5% Ribitol, and an untreated C57 control. Healthy heart and gastrocnemius samples were run at 20, 10, and 5 micrograms of protein per lane as a loading control; mutant and C57 control lysate samples were run with 20 micrograms of protein per lane.

FIG. 6B is a graph depicting total αDG signal in untreated, 5% Ribitol treated, and C57 control samples was quantified using the regions of interest for αDG (˜50-250 kDa) indicated with yellow boxes in FIG. 18A. All data points were normalized to the C57 untreated control.

FIG. 6C is a graph depicting the ratio of glycosylated αDG to total αDG signal in untreated, 5% Ribitol treated, and C57 control samples was quantified using the regions of interest for αDG (˜50-250 kDa) indicated with yellow boxes in FIG. 6A. All data points were normalized to the C57 untreated control.

### DETAILED DESCRIPTION

The disclosed embodiments are based on the unexpected discovery that simultaneous detection of αDG and glycosylated αDG can be used for the quantification of the extent of glycosylation in dystroglycanopathy patient biopsies. Thus, in one embodiment, the present disclosure provides a method of determining a ratio of the amount of a glycosylated form of αDG to the amount of αDG in the sample in a subject with defects in dystroglycan-related genes.

In one aspect, the present disclosure relates to a method of determining an amount of alpha-dystroglycan (αDG) in a sample, determining an amount of the glycosylated form of αDG in a sample, and determining a ratio of the amount of the glycosylated form of αDG to the amount of αDG in a sample.

In some embodiments, determining an amount of alpha-dystroglycan (αDG) in a sample and determining an amount of the glycosylated form of αDG in the sample are performed simultaneously.

In some embodiments, determining an amount of alpha-dystroglycan (αDG) in a sample and determining an amount of the glycosylated form of αDG in the sample comprise performing a Western Blotting analytical method (e.g., as described herein). In some embodiments, a Western Blotting analytical method comprises performing gel electrophoresis.

In some embodiments, the methods of the present disclosure comprise a multiplexed Western Blot method that allows the simultaneous detection of αDG and the glycosylated form of αDG which can be used to compute a ratio of glycosylated αDG to total αDG utilizing the signals obtained from the imaging of the probed membrane. In some embodiments, imaging of the probed membrane comprises contacting the membrane with one or more antibodies.

In some embodiments, determining an amount of αDG in a sample and determining an amount of the glycosylated form of αDG in the sample comprises contacting the sample with one or more antibodies.

In some embodiments, the one or more antibodies comprise one or more monoclonal antibodies. In some embodiments, the one or more antibodies comprise one or more polyclonal antibodies.

In some embodiments, the amount of αDG in a sample was determined by probing a membrane (e.g., a PVDF or nitrocellulose membrane) with AF6868 Human α-dystroglycan antibody. In some embodiments, the antibody was used at a concentration of about 1 microgram per milliliter (μg/mL). In some embodiments, the antibody was used at a concentration of at least about 0.1 μg/mL, 0.5 μg/mL, 1 μg/mL, 2 μg/mL, 3 μg/mL, 4 μg/mL, 5 μg/mL, 6 μg/mL, 7 μg/mL, or greater. In some embodiments, the antibody was used at a concentration of at most about 7 μg/mL, 6 μg/mL, 5 μg/mL, 4 μg/mL, 3 μg/mL, 2 μg/mL, 1 μg/mL, 0.5 μg/mL, 0.1 μg/mL, or lower.

In some embodiments, the amount of glycosylated αDG in a sample was determined by probing a membrane (e.g., a PVDF or nitrocellulose membrane) with anti α-dystroglycan antibody clone IIH6C4. In some embodiments, the antibody was used at a dilution of about 1:1000. In some embodiments, the antibody was used at a dilution of at least about 1:100, 1:500, 1:1000, 1:2000, 1:3000, 1:4000, 1:5000, 1:7000, 1:10,000, 1:12,000, 1:15,000, 1:20,000, or greater. In some embodiments, the antibody was used at a dilution of at most about 1:20,000, 1:15,000, 1:10,000, 1:7000, 1:5000, 1:4000, 1:3000, 1:2000, 1:1000, 1:500, 1:100, or lower.

In some embodiments, the amount of αDG and glycosylated αDG in a sample was determined by contacting the sample with one or more secondary antibodies. In some embodiments, the amount of αDG and glycosylated αDG in a sample was determined by probing a membrane (e.g., a PVDF or nitrocellulose membrane) with IR790 Mouse Anti-Sheep (Light chain) antibody. In some embodiments, the antibody was used at a dilution of about 1:5000. In some embodiments, the antibody was used at a dilution of at least about 1:100, 1:500, 1:1000, 1:2000, 1:3000, 1:4000, 1:5000, 1:7000, 1:10,000, 1:12,000, 1:15,000, 1:20,000, or greater. In some embodiments, the antibody was used at a dilution of at most about 1:20,000, 1:15,000, 1:10,000, 1:7000, 1:5000, 1:4000, 1:3000, 1:2000, 1:1000, 1:500, 1:100, or lower.

In some embodiments, the amount of αDG and glycosylated αDG in a sample was determined by probing a membrane (e.g., a PVDF or nitrocellulose membrane) with IR680 Goat Anti-Mouse (Light chain) antibody. In some embodiments, the antibody was used at a dilution of about 1:5000. In some embodiments, the antibody was used at a dilution of at least about 1:100, 1:500, 1:1000, 1:2000, 1:3000, 1:4000, 1:5000, 1:7000, 1:10,000, 1:12,000, 1:15,000, 1:20,000, or greater. In some embodiments, the antibody was used at a dilution of at most about 1:20,000, 1:15,000, 1:10,000, 1:7000, 1:5000, 1:4000, 1:3000, 1:2000, 1:1000, 1:500, 1:100, or lower.

In some embodiments, the amount of αDG and glycosylated αDG in a sample was determined by visualizing the signals obtained from the imaging of the membrane (e.g., a PVDF or nitrocellulose membrane) that has been contacted with antibodies as described herein. In some embodiments, the membrane was visualized on an Odyssey CLx™ imager (LiCor™) and/or a BioRad ChemiDoc MP analyzer and intensity of a first channel (e.g., 700 nanometers (nm) channel) was recorded. In some embodiments, the membrane was visualized on an Odyssey CLx™ imager and/or a BioRad ChemiDoc MP analyzer and intensity of a second channel (e.g., 800 nm channel) was recorded. In some embodiments, the intensity of first and second channels (e.g., 700 and 800 nm channels) were both recorded. In some embodiments, the intensity of first and second channels (e.g., 700 and 800 nm channels) were recorded simultaneously.

In some embodiments, the amount of αDG and glycosylated αDG in a sample is proportional to the signal at the wavelength corresponding to the secondary antibodies used to detect the primary antibodies. In some embodiments, the amount of αDG is determined by the detected intensity in a first channel, which may be an 800 nm emitting channel. In some embodiments, the amount of glycosylated αDG is determined by the detected intensity in a second channel, which may be a 700 nm emitting channel. The signal intensities of the 800 and 700 nm channels are used to compute the ratio glycosylated αDG to total αDG. This ratio is used to assess the proportion of αDG protein that is glycosylated. This ratio can be compared to ratios corresponding to other samples (e.g., as described herein) to track changes in glycosylation over time, in response to changes in therapy and/or dosing, etc.

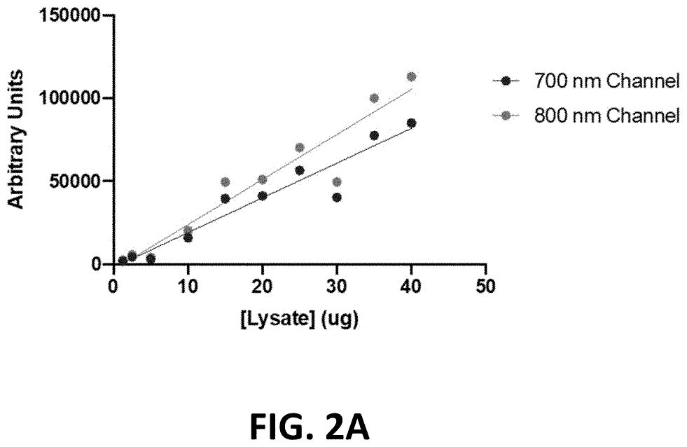

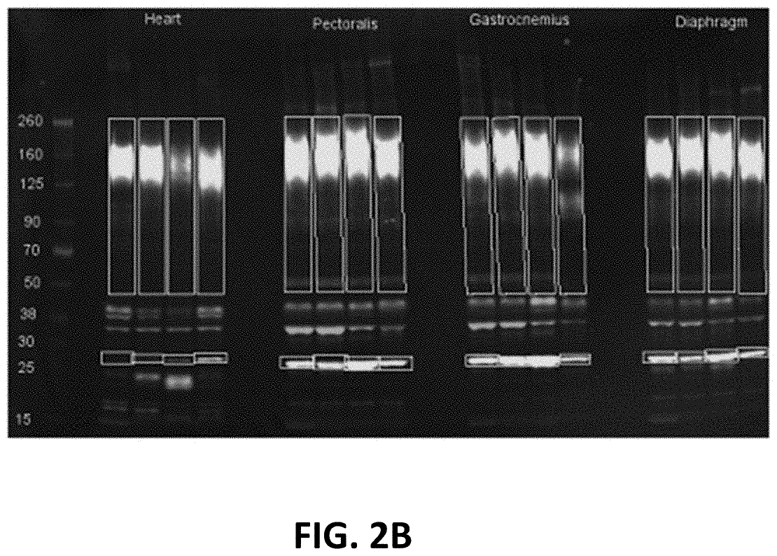

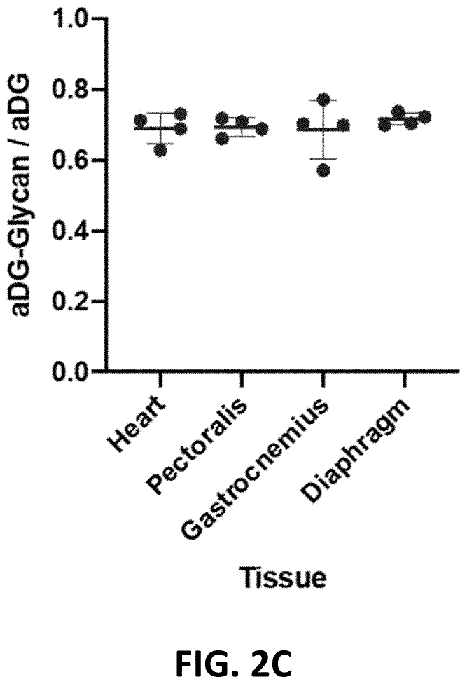

In some embodiments, the intensity of the 700 and 800 nm channels is quantitated by defining an area (e.g., in the form of a rectangle) around each lane and measuring the intensity within that area to measure total protein, as depicted in FIG. 2B. Signal intensities from the 700 and 800 channels were normalized to the measurement of total protein. In some embodiments, the ratio of the normalized 700 nm signal to the normalized 800 nm signal is quantitated.

In some embodiments, the normalized signal intensity of the 800 nm channel is quantified using the formula:

Total αDG/Total protein=Normalized Total αDG

In some embodiments, the normalized signal intensity of the 700 nm channel is quantified using the formula:

Glycosylated αDG/Total protein=Normalized Glycosylated αDG

In some embodiments, the amount of Glycosylated αDG in a sample is determined using the formula:

Normalized Glycosylated αDG/Normalized Total αDG=Glycosylated αDG

In some embodiments, the amount of αDG is determined by selecting the 800 nm channel and defining an area (e.g., in the form of a rectangle) around the region from 50-260 kDa in each lane of a probed membrane and measuring the intensity within that area. In some embodiments, the amount of αDG is determined by selecting 800 nm channel and defining an area (e.g., in the form of a rectangle) around the region from 180-260 kDa, 170-260 kDa, 160-260 kDa, 150-260 kDa, 140-260 kDa, 130-260 kDa, 125-260 kDa, 120-260 kDa, 110-260 kDa, 100-260 kDa, 90-260 kDa, 80-260 kDa, 70-260 kDa, 60-260 kDa, 50-260 kDa, 40-260 kDa, 30-260 kDa, 20-260 kDa, or 10-260 kDa in each lane of a probed membrane and measuring the intensity within that area.

In some embodiments, the amount of glycosylated αDG is determined by selecting the 700 nm channel and defining an area (e.g., in the form of a rectangle) around the region from 125-260 kDa in each lane of a probed membrane and measuring the intensity within that area. In some embodiments, the amount of glycosylated αDG is determined by selecting the 700 nm channel and defining an area (e.g., in the form of a rectangle) around the region from 50-260 kDa in each lane of a probed membrane and measuring the intensity within that area. In some embodiments, the amount of αDG is determined by selecting 800 nm channel and defining an area (e.g., in the form of a rectangle) around the region from 180-260 kDa, 170-260 kDa, 160-260 kDa, 150-260 kDa, 140-260 kDa, 130-260 kDa, 125-260 kDa, 120-260 kDa, 110-260 kDa, 100-260 kDa, 90-260 kDa, 80-260 kDa, 70-260 kDa, 60-260 kDa, 50-260 kDa, 40-260 kDa, 30-260 kDa, 20-260 kDa, or 10-260 kDa in each lane of a probed membrane and measuring the intensity within that area.

In some embodiments, an antibody is used to determine an amount of αDG having a molecular weight of between about 50 kDa and about 300 kDa. In some embodiments, an antibody is used to determine an amount of αDG having a molecular weight of between about 50 kDa and about 260 kDa. In some embodiments, an antibody is used to determine an amount of αDG having a molecular weight of between about 100 kDa and about 260 kDa. In some embodiments, an antibody is used to determine an amount of αDG having a molecular weight of between about 125 kDa and about 260 kDa. In some embodiments, an antibody is used to determine an amount of αDG having a molecular weight of between about 150 kDa and about 260 kDa. In some embodiments, an antibody is used to determine an amount of αDG having a molecular weight of between about 200 kDa and about 260 kDa. In some embodiments, an antibody is used to determine an amount of αDG having a molecular weight of between about 260 kDa and about 300 kDa.

In some embodiments, an antibody is used to determine an amount of αDG having a molecular weight of about 50 kDa, about 70 kDa, about 90 kDa, about 1200 kDa, about 150 kDa, about 180 kDa, about 200 kDa, about 230 kDa, about 260 kDa, about 280 kDa, or about 300 kDa.

In some embodiments, an antibody is used to determine an amount of the glycosylated form of αDG having a molecular weight of between about 50 kDa and about 300 kDa. In some embodiments, an antibody is used to determine an amount of αDG having a molecular weight of between about 50 kDa and about 260 kDa. In some embodiments, an antibody is used to determine an amount of αDG having a molecular weight of between about 100 kDa and about 260 kDa. In some embodiments, an antibody is used to determine an amount of αDG having a molecular weight of between about 125 kDa and about 260 kDa. In some embodiments, an antibody is used to determine an amount of αDG having a molecular weight of between about 150 kDa and about 260 kDa. In some embodiments, an antibody is used to determine an amount of αDG having a molecular weight of between about 200 kDa and about 260 kDa. In some embodiments, an antibody is used to determine an amount of αDG having a molecular weight of between about 260 kDa and about 300 kDa.

In some embodiments, an antibody is used to determine an amount of the glycosylated form of αDG having a molecular weight of about 50 kDa, about 70 kDa, about 90 kDa, about 1200 kDa, about 150 kDa, about 180 kDa, about 200 kDa, about 230 kDa, about 260 kDa, about 280 kDa, or about 300 kDa.

In some embodiments, an antibody of the one or more antibodies is used to determine an amount of αDG and/or an amount of the glycosylated form of αDG having a molecular weight of between about 125 kDa and about 260 kDa.

In some embodiments, determining an amount of alpha-dystroglycan (αDG) in a sample comprises contacting the sample with an anti-αDG antibody and determining an amount of the glycosylated form of αDG in the sample comprises contacting the sample with a matriglycan-specific αDG antibody. In some embodiments, the anti-αDG antibody is AF6868 alpha-dystroglycan antibody. In some embodiments, the matriglycan-specific αDG antibody is IIH6C4 alpha-dystroglycan antibody.

In some embodiments, the anti-αDG antibody detects an N-terminal fragment of αDG having a molecular weight of about 27 kDa.

In some embodiments, the sample is a biopsy sample. In some embodiments, the sample is derived from muscle tissue. In some embodiments, the sample is derived from a subject. In some embodiments, the subject is a human subject.

In some embodiments, the subject has been diagnosed with a dystroglycanopathy (e.g., Walker-Warburg Syndrome, Muscle Eye Brain Disease, Fukuyama Congenital Muscular Dystrophy (CMD), or a Limb Girdle Muscular Dystrophy). In some embodiments, the dystroglycanopathy is limb girdle muscular dystrophy type 2i (LGMD2i), LGMD type 2m (LGMD2m), or LGMD type 2u (LGMD2u). In some embodiments, the subject is suspected to have a dystroglycanopathy. In some embodiments, the subject is suspected to have limb girdle muscular dystrophy type 2i (LGMD2i), LGMD type 2m (LGMD2m), or LGMD type 2u (LGMD2u).

In some embodiments, the method comprises determining that the subject has a dystroglycanopathy (e.g., as described herein). In some embodiments, determining that the subject has dystroglycanopathy is based at least in part on the ratio of the amount of the glycosylated form of αDG to the amount of αDG in a sample.

In some embodiments, the method comprises providing a recommendation to administer a therapeutic agent (e.g., ribitol) to the subject. In some embodiments, providing a recommendation to administer a therapeutic agent (e.g., ribitol) to the subject is based at least in part on the ratio of the amount of the glycosylated form of αDG to the amount of αDG in a sample. In some embodiments, the therapeutic agent is ribitol.

In one aspect, the present disclosure relates to a method of determining the amount of core alpha-dystroglycan (αDG) protein in a sample, wherein the core αDG protein is specifically recognized by an anti-αDG antibody, determining the amount of an additional αDG population in the sample, wherein the additional αDG population is specifically recognized by a matriglycan-specific αDG antibody, and determining a ratio between the amount of the core αDG protein and the amount of the additional αDG population.

In some embodiments, determining the amount of core alpha-dystroglycan (αDG) protein in a sample, wherein the core αDG protein is specifically recognized by an anti-αDG antibody and determining the amount of an additional αDG population in the sample, wherein the additional αDG population is specifically recognized by a matriglycan-specific αDG antibody are performed simultaneously.

In one aspect, the present disclosure relates to a method of providing a first sample from a subject having a first ratio of an amount of a glycosylated form of alpha-dystroglycan (αDG) to an amount of αDG in the first sample, providing a second sample from the subject having a second ratio of an amount of a glycosylated form of αDG to an amount of αDG in the second sample, and determining a difference between the first ratio and the second ratio.

In some embodiments, the first sample was collected from the subject at a first timepoint and the second sample was collected from the subject at a second timepoint, wherein the second timepoint is later than the first timepoint.

In some embodiments, at least 1, at least 3, at least 4, at least 5, at least 6, at least 7, at least 8, at least 9, at least 10, at least 15, at least 25, at least 50, at least, 75 or at least 100 samples are collected from the subject. In some embodiments, the timepoint of each successive sample collection is at a timepoint which is later than the previous sample collection (e.g., a first sample collected at a first timepoint and a second sample collected at a second timepoint, wherein the second time point is later than the first timepoint.)

For example, the first sample is collected from the subject at a first timepoint, a second sample is collected from the subject at a second timepoint, a third sample is collected from the subject at a third timepoint, a fourth sample is collected from the subject at a fourth timepoint, and a fifth sample is collected from the subject at a fifth timepoint. In an embodiment, the second timepoint is later than the first timepoint, the third timepoint is later than the first and second timepoints, the fourth timepoint is later than the first, second, and third timepoints, the fifth timepoint is later than the first, second, third, and fourth timepoints.

In some embodiments, the first sample was collected from the subject prior to the subject undergoing a treatment regimen for a dystroglycanopathy (e.g., Walker-Warburg Syndrome, Muscle Eye Brain Disease, Fukuyama Congenital Muscular Dystrophy (CMD), or a Limb Girdle Muscular Dystrophy).

In some embodiments, the second sample was collected from the subject while the subject is undergoing a treatment regimen for a dystroglycanopathy (e.g., Walker-Warburg Syndrome, Muscle Eye Brain Disease, Fukuyama Congenital Muscular Dystrophy (CMD), or a Limb Girdle Muscular Dystrophy).

In some embodiments, the second sample was collected from the subject after the subject has undergone a treatment regimen for a dystroglycanopathy (e.g., Walker-Warburg Syndrome, Muscle Eye Brain Disease, Fukuyama Congenital Muscular Dystrophy (CMD), or a Limb Girdle Muscular Dystrophy).

In some embodiments, the dystroglycanopathy is limb girdle muscular dystrophy type 2i (LGMD2i).

In some embodiments, a treatment regimen for a dystroglycanopathy (e.g., Walker-Warburg Syndrome, Muscle Eye Brain Disease, Fukuyama Congenital Muscular Dystrophy (CMD), or a Limb Girdle Muscular Dystrophy, such as LGMD2i, LGMD2m, or LGMD2u) comprises administration of ribitol or a form thereof. Ribitol, also known as adonitol or (2R,3s,4S)-Pentane-1,2,3,4,5-pentol, has the chemical structure below and molecule weight of 152.15 g/mol.

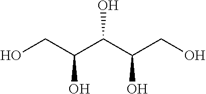

In further embodiments, in place of ribitol, a ribitol derivative may be administered. The ribitol derivative may be a tri-acetylated ribitol; per-acetylated ribitol, ribose; a phosphorylated ribitol (e.g., ribose-5-P); a nucleotide form of ribitol (e.g., a nucleotide-alditol having cytosine or other bases as the nucleobase with 1, 2 or 3 phosphate groups and ribitol as the alditol portion, such as CDP-ribitol or CDP-ribitol-OAc2); or combinations thereof.

In some embodiments, the second timepoint is at least 1 week after the first timepoint. In some embodiments, the second timepoint is at least 2 weeks after the first timepoint. In some embodiments, the second timepoint is at least 1 month after the first timepoint. In some embodiments, the second timepoint is at least 2 months after the first timepoint. In some embodiments, the second timepoint is at least 3 months after the first timepoint. In some embodiments, the second timepoint is at least 4 months after the first timepoint. In some embodiments, the second timepoint is at least 6 months after the first timepoint. In some embodiments, the second timepoint is at least 8 months after the first timepoint. In some embodiments, the second timepoint is at least 10 months after the first timepoint. In some embodiments, the second timepoint is at least 1 year after the first timepoint.

In some embodiments, the method comprises collecting the first sample and/or the second sample from the subject.

In some embodiments, the method comprises determining a relative amount of alpha-dystroglycan (αDG) in the first and/or second sample, determining a relative amount of the glycosylated form of αDG in the first and/or second sample, and determining the first and/or second ratio of the relative amount of the glycosylated form of αDG to the relative amount of αDG in the first and/or second sample.

In some embodiments, determining a relative amount of alpha-dystroglycan (αDG) in the first and/or second sample and determining a relative amount of the glycosylated form of αDG in the first and/or second sample comprises performing a Western Blotting analytical method (e.g., as described herein). In some embodiments, a Western Blotting analytical method comprises performing gel electrophoresis.

In some embodiments, determining a relative amount of alpha-dystroglycan (αDG) in the first and/or second sample and determining a relative amount of the glycosylated form of αDG in the first and/or second sample comprises contacting the first and/or second sample with one or more antibodies.

In some embodiments, the one or more antibodies comprises one or more monoclonal antibodies. In some embodiments, the one or more antibodies comprise one or more polyclonal antibodies.

In some embodiments, the method comprises determining a relative amount of alpha-dystroglycan (αDG) in the first and/or second sample comprises contacting the first and/or second sample with an anti-αDG antibody and determining a relative amount of the glycosylated form of αDG in the first and/or second sample comprises contacting the first and/or second sample with a matriglycan-specific αDG antibody.

In some embodiments, the method further comprises, based at least in part on determining the first and/or second ratio of the relative amount of the glycosylated form of αDG to the relative amount of αDG in the first and/or second sample, providing a recommendation to administer a therapeutic agent to the subject.

In some embodiments, the method further comprises administering a therapeutic agent to the subject. In some embodiments, the therapeutic agent is ribitol or a form thereof.

In some embodiments, the method further comprises, based at least in part on determining the first and/or second ratio of the relative amount of the glycosylated form of αDG to the relative amount of αDG in the first and/or second sample, providing a recommendation to change a dosing regimen of a therapeutic agent for the subject.

In some embodiments, the recommendation comprises a recommendation to increase dosing of the therapeutic agent. In some embodiments, the recommendation comprises a recommendation to decrease dosing of the therapeutic agent.

In some embodiments, the first sample and the second sample are of a same type. In some embodiments, the first sample and the second sample are biopsy samples. In some embodiments, the first sample and the second sample are derived from muscle tissue.

In some embodiments, the first sample and/or the second sample are derived from pectoralis, diaphragm, heart, gastrocnemius, quadricep, or tibialis anterior tissue. In some embodiments, the first sample and/or the second sample are derived from cardiac, smooth, or skeletal muscle tissue. In some embodiments, the first sample and/or the second sample are derived from walls of blood vessels, walls of stomach, ureters, intestines, aorta, iris of the eye, prostate, gastrointestinal tract, respiratory tract, small arteries, arterioles, reproductive tracts, veins, glomeruli of the kidneys, bladder, uterus, arrector pili of the skin, ciliary muscle, sphincter, trachea, bile duct, digestive tract, bronchi, or bronchioles.

The present disclosure also provides a method of assessing the glycosylation of αDG in cells and tissue samples from subjects with dystroglycanopathies to assess longitudinal disease progression. Furthermore, the present disclosure provides a method of evaluating the impact of therapeutics that seek to restore the glycosylation of αDG and may assist in guiding dose selection.

The present disclosure also provides a method of assessing the effect of therapeutic agents that increases the glycosylated form of αDG by assessing biopsy samples before and after treatment with a therapeutic agent. A change in the glycosylated form of αDG and/or the restoration of αDG with treatment could be used to guide dose selection for patients with dystroglycanopathies.

In some embodiments, αDG expression in a subject with at least one mutation in the FKRP gene is increased following ribitol treatment as compared to an untreated subject. In some embodiments, the ratio of glycosylated αDG to total αDG in a subject with at least one mutation in the FKRP gene is increased following ribitol treatment as compared to an untreated subject. In some embodiments, the ratio of glycosylated αDG to total αDG in a subject with at least one mutation in the FKRP gene detects an increase in αDG glycosylation following ribitol treatment as compared to an untreated subject.

The present disclosure provides a method of assessing the glycosylation of αDG in cells and tissue samples from subjects with a disorder associated with (e.g., caused by or resulting from) a mutation in a fukutin related protein (FKRP) gene. The method comprises determining a ratio of the amount of a glycosylated form of αDG to the amount of αDG in the sample from a subject with a disorder associated with a mutation in a fukutin related protein (FKRP) gene disorder associated with a mutation in a fukutin related protein (FKRP) gene in the subject. In some embodiments, the subject harbors a P448L mutation in the FKRP gene. In some embodiments, the subject harbors a L276I mutation in the FKRP gene. In some embodiments, the subject harbors a P448L mutation and/or a L276I mutation in the FKRP gene.

In some embodiments, the present disclosure provides methods of assessing the glycosylation of αDG in cells and tissue samples from subjects to identify subjects for ribitol treatment.

In some embodiments, the present disclosure provides methods of assessing the glycosylation of αDG in cells and tissue samples from subjects to monitor subject's responses to ribitol treatment.

In some embodiments, the present disclosure provides methods of assessing the glycosylation of αDG in cells and tissue samples from subjects to assess and/or amend dosing of subjects with ribitol treatment.

Additionally, the present disclosure provides a method of assessing the glycosylation of αDG in cells and tissue samples from subjects with muscle weakness wherein the subject is a carrier of a mutated FKRP gene and/or with a mutation in a dystroglycan-related gene and/or with a defect in glycosylation of αDG. The muscle weakness can include but is not limited to weakness of skeletal muscle, cardiac muscle and/or respiratory muscle, in any combination, in the subject.

In some embodiments, the disorder associated with muscle weakness can be associated with a defect in glycosylation of αDG, including situations without clear understanding of the underlying causes for the defect.

In some embodiments, a defect in glycosylation of αDG can be associated with a mutated FKRP gene. In some embodiments, a mutated FKRP gene in a subject results in at least about 5%, 10%, 25%, 35%, 40%, 50%, 60%, or 75% reduction in glycosylation of αDG.

In some embodiments, a defect in αDG expression can be associated with a mutated FKRP gene. In some embodiments, a mutated FKRP gene in a subject results in at least about 5%, 10%, 25%, 35%, 40%, 50%, 60%, or 75% reduction in expression of αDG.

In some embodiments, a defect in the ratio of glycosylation of αDG to total αDG can be associated with a mutated FKRP gene. In some embodiments, a mutated FKRP gene in a subject results in at least about 5%, 10%, 25%, 35%, 40%, 50%, 60%, or 75% reduction in the ratio of glycosylation of αDG to total αDG.

In some embodiments, the ratio of glycosylation of αDG to total αDG in a subject provides a biomarker for a recommendation to administer a therapeutic agent to a subject.

In some embodiments, the present disclosure provides a method of assessing the glycosylation of αDG in cells and tissue samples from subjects with muscular dystrophy diseases for which restoration of and/or enhanced glycosylation of αDG would be beneficial and/or therapeutic. A nonlimiting example of a disorder associated with a mutation or loss of function in the FKRP gene is Limb-Girdle Muscular Dystrophy type 2i (LGMD2i). Certain mutations in FKRP are associated with Walker-Warburg Syndrome (WWS) and in congenital muscular dystrophy type 1C (MDC1C). The methods of the present disclosure may also be applied in any disease or disorder associated with metabolism of ribitol and/or any disease or disorder for which ribitol is therapeutically effective.

The methods of this disclosure can be applied to non-muscular dystrophy diseases for which restoration of and/or enhanced glycosylation of αDG would be beneficial and/or therapeutic.

In additional embodiments, the present disclosure provides a method of assessing the glycosylation of αDG in cells and tissue samples from subjects with muscle weakness, e.g., muscle weakness which limits or slows daily activity of the subject. Muscle weakness can imply that a subject is not able to perform the daily activities that a normal person of similar gender, age and other conditions would be expected to be capable of performing. An example is the loss of or lack of ability to climb stairs, run or hold an object for an extended period.

The present disclosure further provides a method of assessing the glycosylation of αDG in cells and tissue samples from subjects with a disorder associated with a defect in glycosylation of αDG, A subject can be suspected of having a defect in glycosylation of αDG if the subject has muscle weakness even in cases where genetic and biochemical analyses of the subject have failed to identify a causative gene defect.

Further provided herein is a method of assessing the glycosylation of αDG in cells and tissue samples from subjects with a disorder associated with a defect in glycosylation of αDG caused by a mutation in the FKRP gene. A mutation in an FKRP gene can be identified by genetic analysis of the nucleic acid of a subject.

In some embodiments, a therapeutic agent for use in the compositions and methods described herein can be ribitol.

In further embodiments, the methods of the disclosure comprise, in place of ribitol, administering a ribitol derivative. The ribitol derivative may be a tri-acetylated ribitol; per-acetylated ribitol, ribose; a phosphorylated ribitol (e.g., ribose-5-P); a nucleotide form of ribitol (e.g., a nucleotide-alditol having cytosine or other bases as the nucleobase with 1, 2 or 3 phosphate groups and ribitol as the alditol portion, such as CDP-ribitol or CDP-ribitol-OAc2); or combinations thereof.

In some embodiments, the disease or disorder is associated with a defect in Fukutin-related protein (FKRP). In some embodiments, the disease or disorder is associated with a defect in fukutin (FKTN). In additional embodiments, a subject has a mutation in a gene encoding fukutin (FKTN), fukutin-related protein (FKRP), or isoprenoid synthase domain-containing protein (ISPD) that causes a partial or complete loss-of-function in FKRP.

In some embodiments, the therapeutic agent of the present disclosure may improve and/or prevent one or more symptoms of disease, including but not limited to limb muscle weakness, e.g., with mild calf and thigh hypertrophy; decreased hip flexion and adduction; decreased knee flexion and ankle dorsiflexion; progressive muscle degeneration; fiber wasting; decreased matriglycan expression; infiltration and accumulation of fibrosis and/or fat in muscle tissues; loss of ambulation. Muscle weakness may involve the diaphragm with varying severity, leading to respiratory failure in a proportion of patients. Cardiac muscle is affected with the most severe and frequent presentation being dilated cardiomyopathy.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description is for the purpose of describing particular embodiments only and is not intended to be limiting. All publications, patent applications, patents and other references mentioned herein are incorporated by reference in their entirety. In case of a conflict in terminology, the present specification is controlling.

As used in the description of the invention and the appended claims, the singular forms “a”, “an”, and “the” are intended to include the plural forms as well, unless the context clearly indicates otherwise.

The term “about,” as used herein when referring to a measurable value such as an amount of dose (e.g., an amount of a fatty acid) and the like, is meant to encompass variations of ±20%, ±10%, ±5%, ±1%, ±0.5%, or even ±0.1% of the specified amount.

“Subject” as used herein includes a mammal, such as primate, mouse, rat, dog, cat, cow, horse, goat, camel, sheep or a pig, preferably a human.

“Treat,” “treating,” or “treatment” as used herein also refers to any type of action or administration that imparts a benefit to a subject that has a disease or disorder, including improvement in the condition of the patient (e.g., reduction or amelioration of one or more symptoms), healing, etc.

The term “effective amount” refers to an amount of an agent (e.g. ribitol) sufficient to have desired biochemical or physiological effect. The term “therapeutically effective amount” refers to an amount of an agent (e.g. ribitol) that is sufficient to improve the condition, disease, or disorder being treated and/or achieved the desired benefit or goal (e.g., control of body weight). Those skilled in the art will appreciate that the therapeutic effects need not be complete or curative, as long as some benefit is provided to the subject. The term “amount” when in reference to αDG or glycosylated αDG refers to the quantification of protein as quantified by detection of a signal obtained from the wavelength corresponding to a secondary antibody used to detect a primary antibody.

The term “enhancement,” “enhance,” “enhances,” or “enhancing” refers to an increase in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or an increase in the specified activity of at least about 5%, 10%, 25%, 35%, 40%, 50%, 60%, 75%, 80%, 90%, 95%, 97%, 98%, 99% or 100%.

The term “inhibit,” “diminish,” “reduce” or “suppress” refers to a decrease in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or a decrease or reduction in the specified activity of at least about 5%, 10%, 25%, 35%, 40%, 50%, 60%, 75%, 80%, 90%, 95%, 97%, 98%, 99% or 100%. These terms are intended to be relative to a reference or control.

The above terms are relative to a reference or control. For example, in a method of detecting glycosylation of αDG in a subject sample, the enhancement is relative to the amount of glycosylation of αDG in a subject sample (e.g., a control subject sample) in the absence of administration of ribitol, CDP-ribitol, ribose and/or ribulose. In another example, in a method of detecting glycosylation of αDG in a sample comprising mutant FKRP, the enhancement is relative to the amount of glycosylation of αDG in a sample (e.g., a reference sample) comprising wild type FKRP.

Also as used herein, “and/or” refers to and encompasses any and all possible combinations of one or more of the associated listed items, as well as the lack of combinations when interpreted in the alternative (or).

Unless the context indicates otherwise, it is specifically intended that the various features described herein can be used in any combination. Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed.

It will also be understood that, as used herein, the terms example, exemplary, illustrative, and grammatical variations thereof are intended to refer to non-limiting examples and/or variant embodiments discussed herein and are not intended to indicate preference for one or more embodiments discussed herein compared to one or more other embodiments.

All publications, patent applications, patents and other references cited herein are incorporated by reference in their entireties for the teachings relevant to the sentence and/or paragraph in which the reference is presented.

Unless the context indicates otherwise, it is specifically intended that the various features described herein can be used in any combination.

Moreover, the present disclosure also contemplates that in some embodiments, any feature or combination of features set forth herein can be excluded or omitted.

### EXAMPLES

The following examples are put forth so as to provide those of ordinary skill in the art with a description of how the compositions and methods described herein may be used, made, and evaluated, and are intended to be purely illustrative of the invention and are not intended to limit the scope of what is regarding as the invention.

### Example 1: Method for the Ratio-Metric Detection of Alpha-Dystroglycan Glycosylation

The following is an illustrative, non-limiting method for the assessment of αDG protein and the matriglycan of αDG in muscle homogenates. A multiplexed Western Blot-based method that reports on the relative O-glycosylation of αDG from muscle biopsies is disclosed herein. The results of 13 LGMD2i patients representing homozygous and compound heterozygous mutations in FKRP are reported. In all patients there was decreased matriglycan expression as compared to healthy human donor tissue. Four of these patients had longitudinal biopsies and there was little change in matriglycan expression from the initial baseline biopsy measurement but significantly reduced matriglycan expression as compared to donor tissue. The semi-quantitative nature of this method may be of use in understanding the longitudinal changes in patients with dystroglycanopathies such as LGMD2i and in assessing the impact of therapeutics directed at increasing the O-glycosylation of αDG.

### Muscle Biopsy

Muscle biopsy samples were collected from tibialis anterior using a fine needle aspirate. In total three passes were collected. Biopsy samples were snap frozen immediately after collection and stored at −80° C. until analysis.

### Healthy Human Tissue

Control healthy human muscle tissue (male and female) was obtained and stored at −80° C. until analysis.

### Murine Mouse Tissue

Murine muscle tissue samples from healthy, P448L, and L276I FRKP mutant mice were stored at −80° C. until analysis.

### Tissue Processing

Tissue samples were processed using a TissueLyzer II™ (Qiagen®). Approximately 250 milligrams (mg) of tissue was placed in a 2 mL microtube with a steel bead and 1 mL of Radioimmunoprecipitation assay buffer (RIPA) buffer supplemented with protease and phosphatase inhibitors. The microtubes were subjected to three cycles of disruption (2 minutes at 25 Hertz (Hz)) in the TissueLyzer II™. Lysates were clarified by centrifugation and the supernatant was transferred to fresh tubes and lysate concentration was determined using a BCA assay (Pierce®).

### SDS-PAGE

Polyacrylamide gel electrophoresis was carried out on 4-20% StainFree™ gels. Tissue lysates as described above were diluted to 20 micrograms (μg)/15 microliters (μL) with RIPA buffer in LiCor™ loading buffer. A total of 20 μg of lysate was loaded per well. Pre-stained protein markers were also loaded onto gel. After electrophoretic separation the gel was washed in water and total protein was imaged using the ChemiDoc™ imaging system (BioRad®).

### Western Blotting

Proteins were transferred to a PVDF membrane using an iBlot™ Gel Transfer Device (Invitrogen®). The membrane was dried and activated with a methanol wash and then rinsed with water. The membrane was blocked with Intercept blocking buffer (LiCor™) and probed with AF6868 Human α-dystroglycan antibody: 1 μg/mL [anti-αDG antibody] (R&D Systems®) and anti α-ystroglycan antibody clone IIH6C4: 1:1000 [anti-glycosylated-αDG antibody] (Millipore®). The primary antibody solution was removed and the washed four times with TBST. The blot was then probed with IR790 Mouse Anti-Sheep (Light chain): 1:5,000 and IR680 Goat Anti-Mouse (Light chain): 1:5,000 secondary antibodies (LiCor™). The secondary antibody solution was removes and the membrane was wash four times with TBST. The membrane was visualized on an Odyssey CLx™ imager (LiCor™) and intensities of the 700 and 800 nm channels were recorded.

### Data Analysis

Analysis was carried on GraphPad Prism™ (version 8). Signal intensities were normalized to total protein and the ratio of 700 nm normalized signal/800 nm normalized signal was determined. Linear fits were assessed where applicable using the linear regression function of Prism™. Statistical significance was determined using an unpaired t-test using Prism™.

1) StainFree gel image is quantitated using ImageLab™ software to analyze the image from the ChemiDoc™ Imaging System. A rectangle is drawn around each lane to measure total protein. Signal intensities were normalized to total protein and the ratio of 700 nm normalized signal/800 nm normalized signal was determined. Linear fits were assessed were applicable using a linear regression function. Statistical significance was determined using an unpaired t-test.

2) α-Dystroglycan is quantitated using ImageStudio™ Software (LiCor™) to analyze the images from the Odyssey:
a. Total α-Dystroglycan is analyzed by selecting 800 channel and drawing a rectangle around the region from 50-260 kDa in each lane.
b. Glycosylated α-Dystroglycan is analyzed by selecting the 700 channel and drawing a rectangle around the region from 50 kDa-260 kDa.

3) Calculations are performed as follows:
a. Normalize Total α-Dystroglycan: Total α-Dystroglycan/Total protein
b. Normalize Glycosylated α-Dystroglycan: Glycosylated α-Dystroglycan/Total protein
c. Glycosylated α-Dystroglycan: Normalized Glycosylated α-Dystroglycan/Normalized Total α-Dystroglycan

### Results

To assess the glycosylation of αDG in cells and tissue samples from patients, a multiplexed Western Blot method was used to simultaneously detect αDG and the glycosylated form of αDG.

Using samples obtained from healthy human donor tissue, the signal intensity (800 nm) observed for total α-dystroglycan in heart, pectoralis, gastrocnemius, and diaphragm tissues displayed a linear regression with respect to μg/lane of total lysate (linear range was observed for ˜1.25-25 μg/lane) (data not shown). The signal intensity (700 nm) observed for glycosylated α-dystroglycan in heart, pectoralis, gastrocnemius, and diaphragm tissues also displayed a linear regression with respect to μg/lane of total lysate (linear range was observed for ˜1.25-25 μg/lane) (data not shown).

Muscle tissue from 4 human donors (2 males and 2 females) was assessed for αDG variability.

Donor 1: 41-year-old African-American male
COD: Cardiopulmonary Arrest following respiratory arrest

| | |
|---|---|
| Medical history: | Type 2 diabetes, hypertension, myocardial infarction |
| BMi: | 40.7 |
| Medications: | Albuterol, Insulin, HTN medication |

Donor 2: 77-year-old Caucasian male
COD: Cardiac - unspecified

| | |
|---|---|
| Medical History: | hypertension, type 2 diabetes, hyperlipidemia, |
| | glaucoma |
| BMI: | 22.1 |
| Medications: | Hypertension and diabetes medications |

Donor 3: 75-year-old Caucasian female
COD: Traumatic subdural hematoma following stroke

| | |
|---|---|
| Medical History: | hypertension, adenoma, arthritis, gastrointestinal reflux |
| | disease |
| BMI: | 34.1 |
| Medications: | blood pressure and thyroid medications |

Donor 4: 61-year-old Caucasian female
COD: Respiratory failure

| | |
|---|---|
| Medical History: | cardiovascular accident, atrial fibrillation |
| BMI: | 27.2 |
| Medications: | Anticoagulant and heart rate regulating medications |

As shown in FIG. 2A, gastrocnemius tissue lysate was serially diluted, and the linearity of each detection channel was plotted as a function of lysate concentration. Four different muscle types (heart, pectoralis, gastrocnemius, and diaphragm) were analyzed to assess the variability of the αDG glycan ratio. As shown in FIG. 2B, the Western Blot membrane displays the merge of both detection channels with regions of interest for αDG (˜50-260 kDa) and a putative N-terminal fragment (˜27 kDa) indicated with yellow boxes. As shown in FIG. 2C, the ratio of glycosylated αDG to total αDG protein was determined using intensities from the detection channels and plotted for each muscle type. The extent of glycosylation of αDG was observed to be consistent among each tissue type.

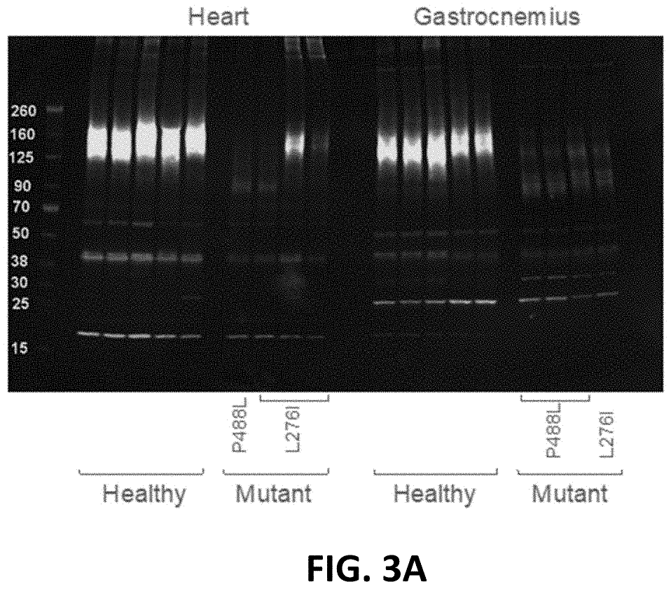

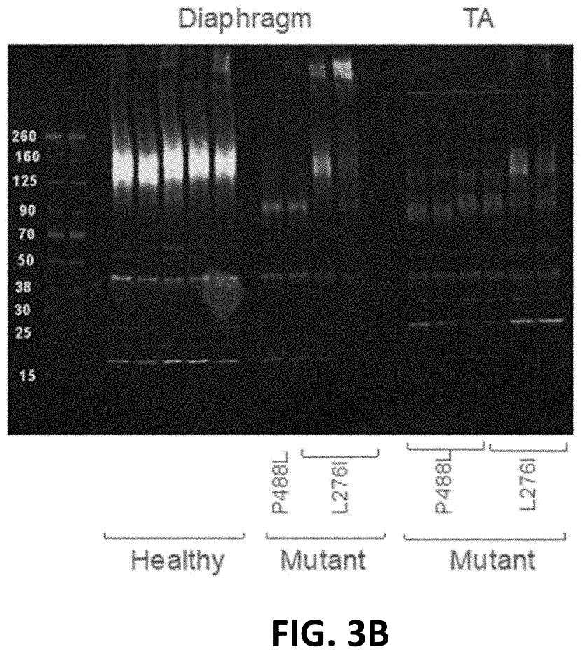

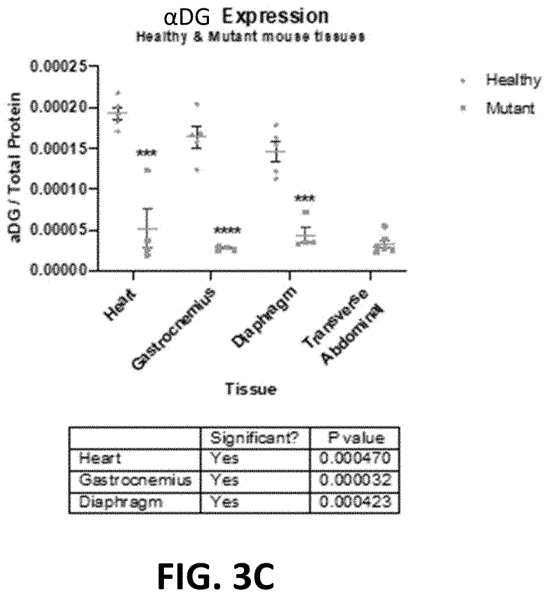

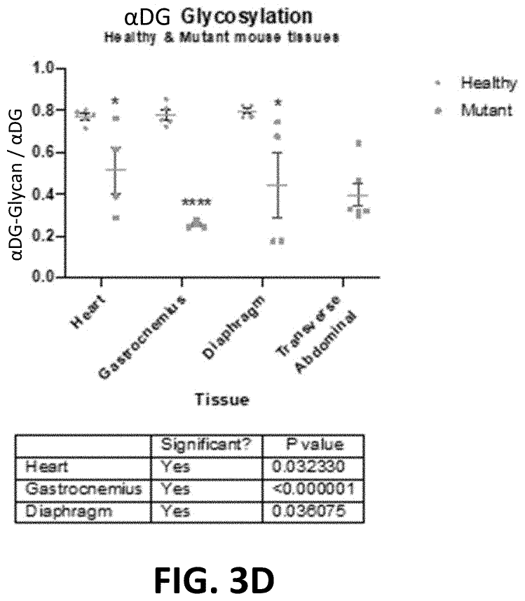

The mutations P448L and L276I in the FKRP protein lead to LGMD2I and result in decreased αDG glycosylation and loss of muscle performance. Samples from rodent models of LGMD2I were analyzed to evaluate decreases in the glycosylation of αDG in muscle tissue. As shown in FIG. 3A, the Western Blot membrane displays total αDG and glycosylated αDG in healthy and mutant mouse heart and gastrocnemius tissues. In FIG. 3B, the Western Blot membrane displays total αDG and glycosylated αDG in healthy and mutant mouse diaphragm and tibialis anterior (TA) tissues. As shown in FIG. 3C, the ratio of total αDG to total protein was determined using intensities in the ˜50-260 kDa range and plotted for each muscle type. In FIG. 3D, the ratio of glycosylated αDG to total αDG was determined using intensities in the ˜50-260 kDa range and plotted for each muscle type. Both αDG expression and glycosylation of αDG was observed to be significantly decreased in all tissue types from mutant animals. This data demonstrated that decreases in αDG were present in mice harboring mutations in FKRP that are known to cause LGMD2I in humans.

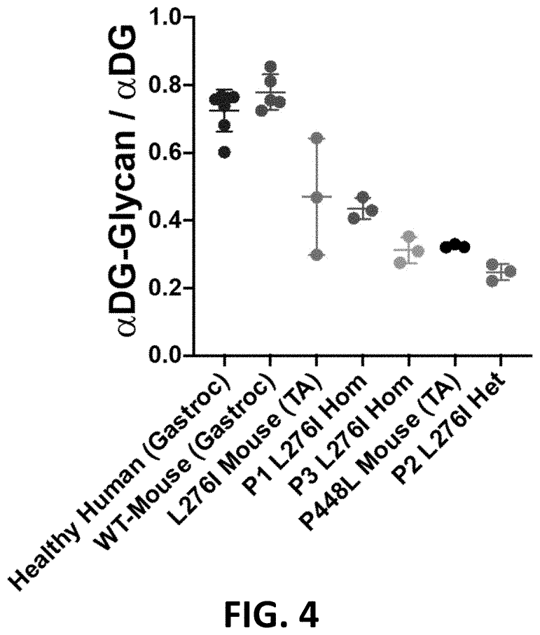

As shown in FIG. 4, the ratio of glycosylated αDG to total αDG was assessed in gastrocnemius muscle tissue from a healthy human donor (Healthy Human) and a wild type mouse (WT-Mouse), TA muscle tissue from a L276I FKRP mutant mouse model (L276I Mouse) and a P448L mutant mouse model (P448L Mouse), and three LGMD2I patients containing a L276I FRKP mutation (two homozygous: [P1 L276I and P3 L276I] and one compound heterozygous: [P2 L276I]). The data show an αDG glycosylation defect in all samples from subjects with FKRP mutations as compared to healthy and wild type subjects. The αDG glycosylation defect was exhibited though statistically significant lower ratios of glycosylated αDG to total αDG in all samples from subjects with FKRP mutations as compared to healthy and wild type subjects (Table 1). Table 1 summarizes the mean and standard deviation of the measurements and statistical significance.

TABLE 1

| | | | P Unpaired |
|---|---|---|---|
| Sample | Mean Ratio | SD | t-test |
| Healthy Human (Gastroc) | 0.72 | 0.06 | |
| WT-Mouse (Gastroc) | 0.78 | 0.03 | |
| L276I Mouse (TA) | 0.47 | 0.17 | 0.10 |
| P1 L276I Hom | 0.43 | 0.03 | <0.0001 |
| P3 L276I Hom | 0.31 | 0.04 | <0.0001 |
| P448L Mouse (TA) | 0.32 | 0.005 | <0.0001 |
| P2 L276I Het | 0.25 | 0.02 | <0.0001 |

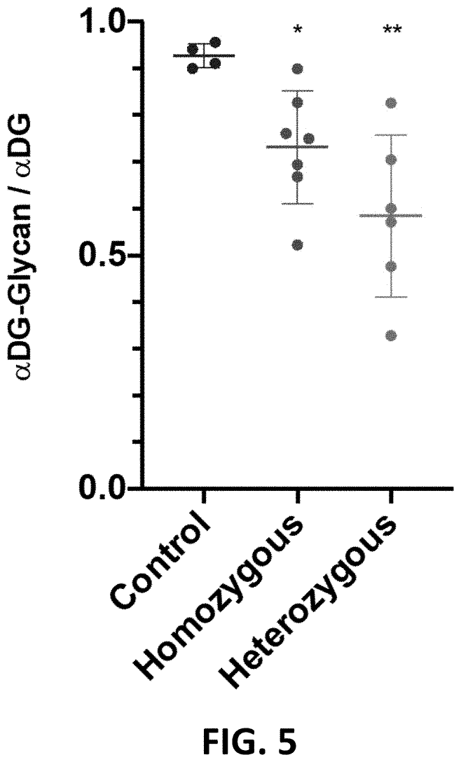

Samples from human LGMD2I patients with mutation L276I were analyzed for αDG glycosylation ratio (FIG. 5).

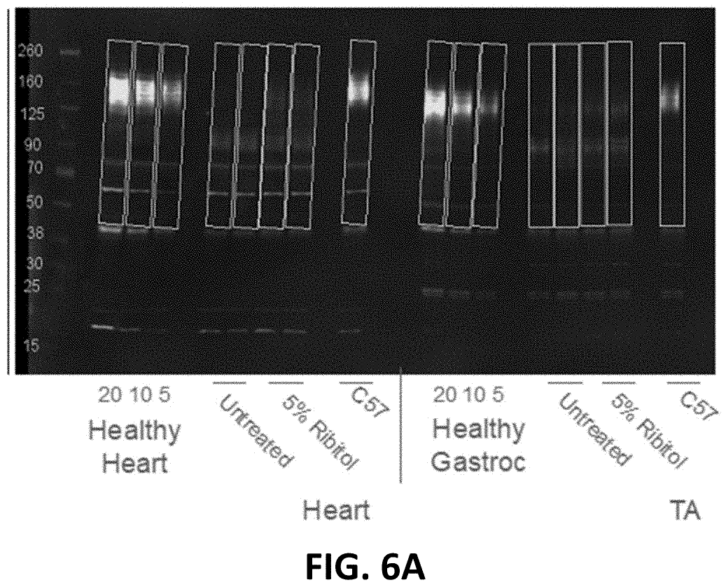

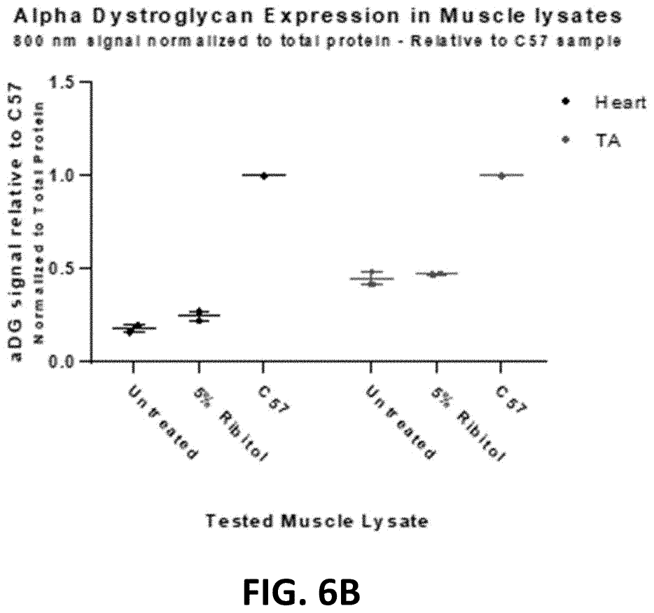

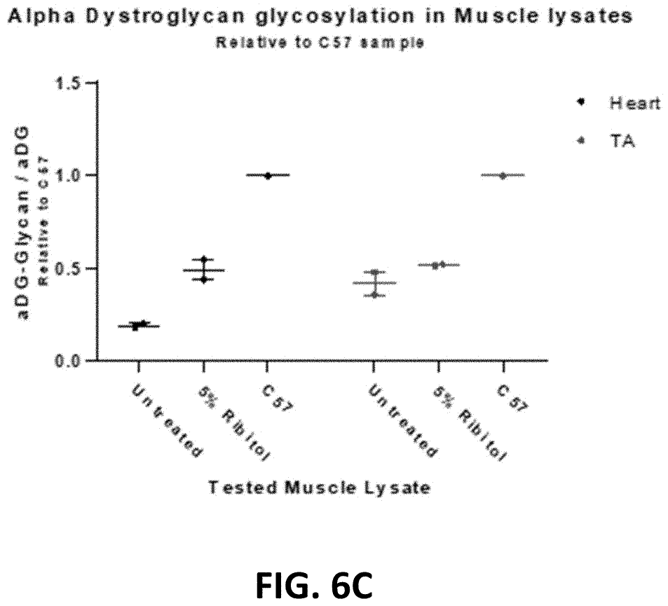

Samples from P448L mutant FKRP mice were analyzed to evaluate glycosylation of αDG following restorative therapy. As shown in FIG. 6A, the Western Blot membrane displays the merge of both detection channels with regions of interest for αDG (˜50-250 kDa) indicated with yellow boxes. The samples included healthy heart muscle tissue, healthy gastrocnemius muscle tissue, and P448L mutant FKRP heart and gastrocnemius tissues that were untreated or treated with 5% Ribitol, and an untreated C57 control for normalization.

As shown in FIG. 6B, total αDG signal in untreated, 5% Ribitol treated, and C57 control samples was quantified using the regions of interest for αDG (˜50-250 kDa) indicated with yellow boxes in FIG. 6A. In FIG. 6C, the ratio of glycosylated αDG to total αDG signal in untreated, 5% Ribitol treated, and C57 control samples was quantified using the regions of interest for αDG indicated in FIG. 6A. The data show that αDG expression is increased in 5% Ribitol treated P488L mice as compared to untreated P488L mice in both heart muscle and TA lysates (FIG. 6B). The data also show that the ratio of glycosylated αDG to total αDG is increased in 5% Ribitol treated P488L mice as compared to untreated P488L mice in both heart muscle and TA lysates (FIG. 6C). The data demonstrate that this assay is capable of detecting increases in αDG glycosylation with a restorative therapy.

### CONCLUSIONS

A multiplexed Western Blot method to detect the radiometric extent of αDG glycosylation relative to total αDG from muscle lysates that can be applied to human biopsy samples was demonstrated. The detection signals show linearity with lysate concentration.

Analysis of several muscle types from human donors demonstrated that the αDG ratio is similar across different muscle types. Tibialis anterior from two murine models of LGMD2i were analyzed using method of the present disclosure and a decrease in the glycation of αDG was observed in both FKRP mutant mouse models with the largest reduction observed in the P448L FKRP mutation. Three biopsies from LGMD2i patients harboring an L276I mutation displayed statistically significant reductions in αDG glycosylation. Samples from several LGMD2i patients were analyzed and statistically significant reduction in αDG glycation was observed. Compound heterozygous patient showed a great loss in αDG glycosylation.

The methods of the present disclosure may be used understanding the longitudinal changes in patients with LGMD2i and in assessing the impact of therapeutics directed at increasing the O-glycosylation of αDG.

### Abbreviations

| Abbreviation | Definition |
|---|---|
| αDG | alpha-dystroglycan |
| βDG | beta-dystroglycan |
| AF6868 | Sheep Anti-Human Dystroglycan Antigen Polyclonal |
| | Antibody |
| BLQ | below limit of quantitation |
| C57 | C57BL/6 is a common inbred strain of laboratory mouse |
| | used as “genetic background” for genetically modified |
| | mice for use as models of human disease |
| CDP-ribitol | Cytidine 5-diphosphotase ribitol |
| CMD | Congenital muscular dystrophy |
| DAG1 | dystroglycan 1 |
| DGC | dystrophin-associated glycoprotein complex |
| FKRP | Fukutin-related protein |
| FKTN | Fukutin |
| GMR | Geometric mean ratio |
| IIH6C4 | monoclonal anti-α-dystroglycan antibody |
| ISPD | isoprenoid synthase domain containing protein |
| LGMD | limb girdle muscular dystrophy |
| LGMD2i | limb girdle muscular dystrophy type 2i |
| rib | ribitol |
| Ribitol 5P | ribitol-5-phosphate |
| TA | Tibialis anterior |
| TMEM | transmembrane protein 5 |

What is claimed is:

1. A method, comprising:
a) determining an amount of alpha-dystroglycan (αDG) in a sample;
b) determining an amount of the glycosylated form of αDG in the sample; and
c) determining a ratio of the amount of the glycosylated form of αDG to the amount of αDG in the sample,

2. The method of claim 1, wherein (a) and (b) are performed simultaneously.

3. The method of claim 1, wherein (a) and/or (b) comprise performing a Western Blotting analysis.

4. The method of claim 1, wherein (a) and/or (b) comprises contacting the sample with one or more antibodies.

5. The method of claim 4, wherein an antibody of the one or more antibodies is used to determine an amount of αDG and/or an amount of the glycosylated form of αDG having a molecular weight of between about 50 kiloDaltons (kDa) and about 260 kDa.

6. The method of claim 1, wherein the sample is a muscle tissue biopsy sample.

7. The method of claim 6, wherein the sample is derived from a subject.

8. The method of claim 7, wherein the subject has been diagnosed with a dystroglycanopathy.

9. The method of claim 7, wherein the subject has been diagnosed with limb girdle muscular dystrophy type 2i (LGMD2i).

10. The method of claim 1, further comprising, based at least in part on (c), determining that a subject has a dystroglycanopathy.

11. The method of claim 10, wherein the subject is determined to have limb girdle muscular dystrophy type 2i (LGMD2i).

12. The method of claim 1, further comprising, based at least in part on (c), providing a recommendation to administer a therapeutic agent to a subject.

13. The method of claim 12, wherein the therapeutic agent is ribitol or a form thereof.

14. A method, comprising:
a) providing a first sample from a subject having a first ratio of an amount of a glycosylated form of alpha-dystroglycan (αDG) to an amount of αDG in the first sample;
b) providing a second sample from the subject having a second ratio of an amount of a glycosylated form of αDG to an amount of αDG in the second sample;
c) determining a difference between the first ratio and the second ratio.

15. The method of claim 14, wherein the first sample was collected from the subject at a first timepoint and the second sample was collected from the subject at a second timepoint, wherein the second timepoint is later than the first timepoint.

16. The method of claim 14, wherein the first sample was collected from the subject prior to the subject undergoing a treatment regimen for a dystroglycanopathy, and wherein the second sample was collected from the subject while the subject is undergoing a treatment regimen for a dystroglycanopathy.

17. The method of claim 16, wherein the dystroglycanopathy limb girdle muscular dystrophy type 2i (LGMD2i).

18. The method of claim 16, wherein the treatment regimen comprises administration of ribitol or a form thereof.

19. The method of claim 14, further comprising:
i) determining a relative amount of alpha-dystroglycan (αDG) in the first and/or second sample;
ii) determining a relative amount of the glycosylated form of αDG in the first and/or second sample; and
iii) determining the first and/or second ratio of the relative amount of the glycosylated form of αDG to the relative amount of αDG in the first and/or second sample.

20. A method, comprising:
a) determining the amount of core alpha-dystroglycan (αDG) protein in a sample, wherein the core αDG protein is specifically recognized by an anti-αDG antibody;
b) determining the amount of an additional αDG population in the sample, wherein the additional αDG population is specifically recognized by a matriglycan-specific αDG antibody; and
c) determining a ratio between the amount of the core αDG protein and the amount of the additional αDG population.
